(19) United States
(12) Patent Application Publication
(10) Pub. No.: US 2021/0240931 A1
(43) Pub. Date: Aug. 5, 2021

(54) VISUAL QUESTION ANSWERING USING ON-IMAGE ANNOTATIONS

(71) Applicant: KONINKLIJKE PHILIPS N.V., EINDHOVEN (NL)

(72) Inventors: Oladimeji Feyisetan FARRI, Yorktown Heights, NY (US); Sheikh Sadid AL HASAN, Cambridge, MA (US); Yuan LING, Somerville, MA (US)

(21) Appl. No.: 17/049,642

(22) PCT Filed: Apr. 29, 2019
(86) PCT No.: PCT/EP2019/060977

§ 371 Date: Oct. 22, 2020

Related U.S. Application Data
(60) Provisional application No. 62/664,443, filed on Apr. 30, 2018.

(51) Int. Cl. G06F 40/279; G06F 3/16; G06F 40/30; G06N 20/00

(52) U.S. Cl. CPC G06F 40/279; G06F 3/16; A61B 5/055; G06N 20/00; G06F 40/30

(57) ABSTRACT

Techniques described herein relate to visual question answering (“VQA”) using trained machine learning models. In various embodiments, a VQA machine learning model may be trained using the follow operations: obtaining (302) a corpus of digital images, each respective digital image (232) including on-image annotation(s) (234) that identify pixel coordinate(s) on the respective digital image; obtaining (304) question-answer pair(s) associated with each of the digital images; generating (306) training examples, each including a respective digital image of the corpus, including the associated on-image annotations, and the associated question-answer pair(s); and for each respective training example of the plurality of training examples: applying (312) the respective training example as input across a machine learning model to generate a respective output; and training (314) the machine learning model based on comparison of the respective output with an answer of the question-answer pair(s) of the respective training example.

### TECHNICAL FIELD

Various embodiments described herein are directed generally to health care. More particularly, but not exclusively, various methods and apparatus disclosed herein relate to visual question answering for various contexts, such as health care.

### BACKGROUND

With the ongoing drive for improved patient engagement, including making electronic medical records available via patient portals, patients are now more than ever able to review various data associated with their healthcare utilization. Such access can, with the guidance of their health care providers, help patients better understand their conditions. Furthermore, patients may have questions about the morphology/physiology and/or disease status of their medical conditions—and they may not necessarily be willing to pay significant amounts for a separate office or hospital visit to address such questions. Since patients may not be equipped with the knowledge to understand all that is represented in a medical, they often turn to search engines to disambiguate complex terms or obtain answers to confusing aspects of the medical image. However, results from search engines may be non-specific, erroneous and misleading, or overwhelming in terms of the volume of information.

The ability of patients to comprehend their health-related information, including annotations in medical images, is helpful to equip them with the knowledge and answers they need to effectively manage their healthcare and act on providers' recommendations. Unfortunately, technology to support question answering for medical images (especially outside the clinician-patient encounter) is not readily available. Visual question answering (“VQA”) combines natural language processing and computer vision to enable people (“users”) to ask questions about digital images that can be answered automatically, e.g., using trained machine learning models. However, known VQA techniques have not yet been used to handle images as complex as medical images that are produced using modalities such as MRI, CT, X-ray, etc. Moreover, VQA models may require training data (e.g., digital images) to be manually segmented prior to training, which is time-consuming and resource intensive.

### SUMMARY

The present disclosure is directed to methods and apparatus for visual question answering for various contexts, such as health care. For example, in some embodiments, on-image annotations that already exist on digital images, especially in the medical context, may be leveraged to segment training data so that it can be used to train machine learning models. These models may then be usable in the VQA context to answer questions about images.

In the context of the examples described herein, the digital images may be medical images obtained using one or more of magnetic resonance imaging (“MRI”), computed tomography (“CT”) scanning, x-ray imaging, etc. The people (or “users”) posing the questions may be patients, clinicians, insurance company personnel, medics, triage personnel, medical students, and so forth. However, techniques described are not limited to the medical context of the examples described herein. Rather, techniques described herein may be applicable in a wide variety of scenarios in which visual question answering is used, such as engineering (e.g., answering questions about components depicted in product images), architecture (e.g., answer questions about architectural features depicted in digital images), biology (e.g., answer questions about features of digital images of slides), topology, topography, surveillance (e.g., interpreting satellite images), etc.

As noted in the background, in the medical context patients are being provided with steadily-increasing access to their medical records, including medical images. Rather than patients haphazardly using search engines to self-diagnose or seek additional information, or being compelled to engage directly with busy medical personnel, techniques described herein provide patients with the ability to ask questions about their specific medical images, and have answers automatically generated in a consistent manner. To this end, in various embodiments, one or more machine learning models, such as a pipeline of machine learning models, may be trained to generate answers to questions posed by people about digital images.

In some embodiments, the machine learning model may take the form of a neural network architecture that includes an encoder portion and a decoder portion. While many variations are possible, in some embodiments, the encoder portion includes a convolutional neural network with one or more attention layers (described below), and the decoder portion includes a recurrent neural network (“RNN”), e.g., that may be one or more long short-term memory (“LSTM”) units and/or one or more gated recurrent units (“GRU”). In some embodiments, the decoder may take the form of an “attention-based” RNN.

In various embodiments, the machine learning model may be trained using training data that includes (i) digital images, (ii) on-image annotations of the digital images, and (iii) pairs of questions and answers (also referred to as “question-answer pairs”) in textual form. For example, a given training example in the medical VQA context may include a medical digital image with one or more on-image annotations identifying one or more features of medical significance, as well as a question posed about one or more of the medically-significant features and an answer to the question (in some embodiments, multiple pairs of question-answers, such as greater than twenty, may be provided for each medical image). In some embodiments, the question may be associated with the targeted feature by way of a label or token that “links” the question with the on-image annotation that describes the targeted feature.

During training, each training example may be applied as input across the machine learning model to generate output. More specifically, some or all of the constituent components of the training example (e.g., <image, on-image annotations, question, answer>) may be encoded by the encoder portion into a semantically-rich encoding that encapsulates the constituent components as, for instance, a reduced-dimensionality feature vector (sometimes referred to as an “embedding”). For example, in some embodiments, the image and on-image annotations may be encoded by a portion of the encoder taking the form of a convolutional neural network. Meanwhile, the textual data forming the question-answer pair (or in some cases, only the question) may be encoded by another portion of the encoder that takes the form of one or more long short-term memory (“LSTM”) or gated recurrent unit (“GRU”) components. In some embodiments, these two encoded may be combined, e.g., concatenated.

In various embodiments, the decoder portion of the architecture may attempt to decode the (joint) encoding to recreate selected portions of the input training example, such as the answer portion of question-answer pair. In some embodiments, the answer portion of the question-answer pair may be provided to the decoder portion, e.g., as a label. In some embodiments, a difference (or “loss function”) between the output of the decoder portion and the answer portion may then be optimized to improve the model's accuracy, e.g., using techniques such as stochastic gradient descent leveraging back propagation etc. In other embodiments, the decoder may embed the joint encoding into a higher dimensionality space that essentially maps the encoding to the answer of the question-answer pair.

Once the machine learning model is trained, it may be used to answer users' questions about images (even if unannotated). For example, a mother-to-be may receive an (annotated or unannotated) ultrasound image of her fetus relatively early in pregnancy. Because the fetus may not yet be readily recognizable (at least by a layperson) as human, the mother-to-be may be curious to learn more about particular features in the ultrasound image. Accordingly, the mother-to-be may formulate a free-form natural language question, and her question, along with the image, may be applied as input across the trained machine learning model to generate output. In various embodiments, the output may be indicative of an answer to her question.

For example, suppose she asks a “factoid” question that seeks specific factual information, such as “what is this image depicting?” In various embodiments, the answer may be data indicative of an anatomical feature or view. In some such embodiments, the answer may be selected from a plurality of outputs generated by the trained model, with each output corresponding to a particular candidate anatomical feature or view and being associated with a probability that the candidate anatomical feature or view is the correct answer.

Additionally or alternatively, suppose the mother-to-be asks a more complicated question, such as “what are the white appearances around where the heart is?” This “non-factoid” is relatively complex, and may seek more general information and/or guidance. In some such embodiments, the trained machine learning model may generate output that indicates a one or more semantic concepts detected in the image. In some embodiments, the decoder portion of the trained model may use a hierarchical co-attention-based mechanism to attend to the question context, e.g., to identify semantic concepts baked into the question, and associate these concepts with features of the ultrasound image. Thus, the output may include, for instance, one or more candidate anatomical structures that the mother-to-be may be referring to. In some embodiments, natural language output may be generated using these outputs, so that the mother-to-be can be presented with an answer such as “They could be x, or y, or z.” Or, in some embodiments, the best answer provided as part of a training example during training of the underlying models may be selected and output to the user.

Generally, in one aspect, a non-transitory computer-readable medium may store a machine learning model, and the model may be trained using the following process: obtaining a corpus of digital images, wherein each respective digital image of the corpus includes one or more on-image annotations, each on-image annotation identifying at least one pixel coordinate on the respective digital image; obtaining at least one question-answer pair associated with each of the digital images of the corpus; generating a plurality of training examples, wherein each training example includes a respective digital image of the corpus, including the associated on-image annotations, and the associated at least one question-answer pair; for each respective training example of the plurality of training examples: applying the respective training example as input across a machine learning model to generate a respective output, wherein the machine learning model comprises an encoder portion and a decoder portion, wherein the encoder portion includes an attention layer that is configured to focus the encoder portion on a region of the digital image of the respective training example, wherein the region is selected based on the at least one pixel coordinate identified by the on-image annotation of the digital image of the respective training example; training the machine learning model based on comparison of the respective output with the answer of the at least one question-answer pair of the respective training example.

In various embodiments, the encoder portion may include or take the form of a convolutional neural network. In various embodiments, the decoder portion may include or take the form of a recurrent neural network. In various embodiments, the decoder portion may be configured to decode the answer of the at least one question-answer pair of the respective training example based on an encoding generated using the digital image, at least one pixel coordinate, and the question-answer pair of the respective training example as input. In various embodiments, the encoder portion may include or take the form of one or more bidirectional long short term memory networks and/or gated recurrent units. In various embodiments, the corpus of digital images may include medical images obtaining using one or more of magnetic resonance imaging, computed tomography.

In another aspect, a method may include: obtaining a digital image; receiving, from a computing device operated by a user, a free-form natural language input; analyzing the free-form natural language input to identify data indicative of a question by the user about the digital image; applying the data indicative of the question and the digital image as input across a machine learning model to generate output indicative of an answer to the question by the user; and providing, at the computing device operated by the user, audio or visual output based on the generated output. In various embodiments, the machine learning model may include an encoder portion and a decoder portion that are trained using a plurality of training examples. In various embodiments, each respective training example may include: a digital image that includes one or more on-image annotations that are used to focus attention of the encoder portion on a region of the digital image; and a question-answer pair associated with the digital image of the respective training example. In various embodiments, the decoder portion may decode the answer of the at least one question-answer pair of the respective training example based on an encoding generated using the digital image, the one or more on-image annotations, and the question-answer pair of the respective training example as input.

In yet another aspect, a method may include: obtaining a corpus of digital images, wherein each respective digital image of the corpus includes one or more on-image annotations, each on-image annotation identifying at least one pixel coordinate on the respective digital image; obtaining at least one question-answer pair associated with each of the digital images of the corpus; generating a plurality of training examples, wherein each training example includes a respective digital image of the corpus, including the associated on-image annotations, and the associated at least one question-answer pair; for each respective training example of the plurality of training examples: applying the respective training example as input across a machine learning model to generate a respective output, wherein the machine learning model comprises an encoder portion and a decoder portion, wherein the encoder portion includes an attention layer that is configured to focus the encoder portion on a region of the digital image of the respective training example, wherein the region is selected based on the at least one pixel coordinate identified by the on-image annotation of the digital image of the respective training example; training the machine learning model based on comparison of the respective output with the answer of the at least one question-answer pair of the respective training example.

In addition, some implementations include one or more processors of one or more computing devices, where the one or more processors are operable to execute instructions stored in associated memory, and where the instructions are configured to cause performance of any of the aforementioned methods. Some implementations also include one or more non-transitory computer readable storage media storing computer instructions executable by one or more processors to perform any of the aforementioned methods.

It should be appreciated that all combinations of the foregoing concepts and additional concepts discussed in greater detail below (provided such concepts are not mutually inconsistent) are contemplated as being part of the inventive subject matter disclosed herein. In particular, all combinations of claimed subject matter appearing at the end of this disclosure are contemplated as being part of the inventive subject matter disclosed herein. It should also be appreciated that terminology explicitly employed herein that also may appear in any disclosure incorporated by reference should be accorded a meaning most consistent with the particular concepts disclosed herein.

### BRIEF DESCRIPTION OF THE DRAWINGS

In the drawings, like reference characters generally refer to the same parts throughout the different views. Also, the drawings are not necessarily to scale, emphasis instead generally being placed upon illustrating various principles of the embodiments described herein.

FIG. 1 illustrates an example environment which selected aspects of the present disclosure may be implemented, in accordance with various embodiments.

FIG. 2 depicts an example process flow for training and using VQA models, in accordance with various embodiments.

FIG. 3 depicts an example method for training one or more models to facilitate VQA, in accordance with various embodiments described herein.

FIG. 4 depicts an example method for utilizing one or more models training using the method of FIG. 3, in accordance with various embodiments.

FIG. 5 depicts an example computing system architecture.

### DETAILED DESCRIPTION

With the ongoing drive for improved patient engagement and access to electronic medical records via patient portals, patients are now more than ever able to review various data associated with their healthcare utilization, including medical images generated using CT, MRI, x-ray, ultrasound, etc. However, there is a risk that without guidance, information discovered by patients on their own may be inaccurate, misleading, etc. Additionally or alternatively, medical images can be interpreted by different clinicians in different ways. A junior radiologist or nurse practitioner may interpret an x-ray image feature one way, but may be uncertain and desire confirmation from someone else, such as e.g., a senior radiologist. In view of the foregoing, various embodiments and implementations of the present disclosure are directed to visual question answering for various contexts, such as health care.

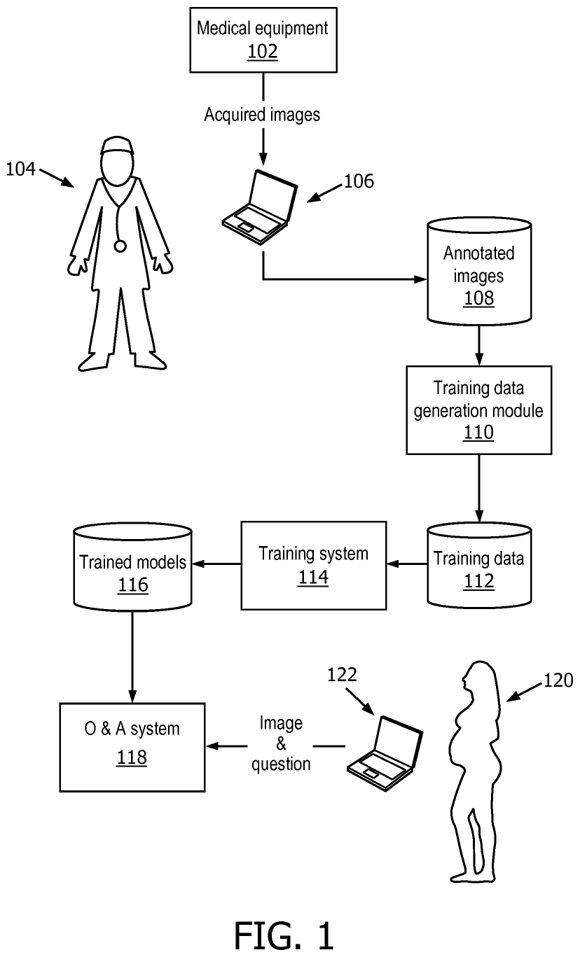

Referring to FIG. 1, an example environment is depicted schematically, showing various components that may be configured to perform selected aspects of the present disclosure. One or more of these components may be implemented using any combination of hardware or software. For example, one or more components may be implemented using one or more microprocessors that execute instructions stored in memory, a field-programmable gate array (“FPGA”), and/or an application-specific integrated circuit (“ASIC”). The connections between the various components represent communication channels that may be implemented using a variety of different networking technologies, such as Wi-Fi, Ethernet, Bluetooth, USB, serial, etc. In embodiments in which the depicted components are implemented as software executed by processor(s), the various components may be implemented across one or more computing systems that may be in communication over one or more networks (not depicted).

In this example, medical equipment 102 may be configured to acquire medical images depicted various aspects of patients. It is not essential that all such images be captured as digital images, and some images, such as x-ray images captured using older machines, may be in analog form. However, techniques described herein are designed to operate on digital images, so it may be assumed that images analyzed and/or processed using techniques described herein are digital images, whether they were natively captured in digital or converted from analog to digital. In various implementations, medical equipment 102 may take various forms, such as a CT scanner, an MRI capture system, an x-ray, an ultrasound, etc.

Digital images acquired by medical equipment 102 may be viewed by a clinician 104 using one or more client devices 106. Client device(s) 106 (and other client devices described herein) may include, for example, one or more of: a desktop computing device, a laptop computing device, a tablet computing device, a mobile phone computing device, a computing device of a vehicle of the user (e.g., an in-vehicle communications system, an in-vehicle entertainment system, an in-vehicle navigation system), a smart appliance such as a smart television, and/or a wearable apparatus of the user that includes a computing device (e.g., a watch of the user having a computing device, glasses of the user having a computing device, a virtual or augmented reality computing device). Additional and/or alternative client computing devices may be provided.

In various embodiments, clinician 104, which may be a doctor, nurse, technician, etc., may operate client device 106 to add on-image annotations to the images and store them in annotated images database 108 (which may be integral with client device 106 and/or separate therefrom, e.g., as part of the “cloud”). “On-image” annotations may include text, symbols, arrows, etc., that may be superimposed on top of a digital image. One type of on-image annotation many, including expecting parents, are familiar with are those often superimposed onto ultrasound images of fetuses in utero. Other types of annotations may be applied by clinician 104 for a variety of reasons, such as diagnoses, observation, etc. For example, in a CT scan, clinician 104 (e.g., a radiologist) might add on-image annotations that identify medically-significant features, such as anatomical structures, lesions, tumors, etc.

In some implementations, on-image annotations may be “baked” into the digital images. For example, pixels covered by the on-image annotations may have their values replaced and/or augmented so that the on-image annotations are visible over the image. Additionally or alternatively, in some embodiments, on-image annotations may be provided separately, e.g., as metadata, and a software application that is usable to view the annotated images may superimpose the on-image annotations over the underlying rendered medical image at runtime. For purposes of the present disclosure, it is the position of the on-image annotations, e.g., the x and y coordinates, that are particular useful for training VQA models.

Notably, as a matter of routine medical practice, clinicians and other medical personnel already add on-image annotations to medical images. Accordingly, techniques described herein seek to leverage those on-image annotations to facilitate improved visual question answering. This may enable patients to obtain answers to questions they have about medical images to which they are being provided ever-increasing access. Additionally or alternatively, medical personnel such as nurses or nurse practitioners, or even medical students, to ask questions about medical images, e.g., for diagnostic purposes, or at least to identify potential issues.

To these ends, in some embodiments, a training data generation module 110 may be configured to generate training data 112 based on annotated images in database 108. The training data 112 may then be used by a training system 114 to train one or more machine learning models 116. The trained machine learning models 116 may then be used by a question and answer (“Q&A”) system 118 to answer questions submitted by users (e.g., patient 120) using client devices 122.

In various embodiments, training data generation module 110 may be operated by one or more experts (not depicted) who are tasked with formulating questions and answers about each annotated medical image. These experts may be medical personal, researchers, etc., who have sufficient knowledge and/or experience to be able to intelligently interpret medical images and their accompanying on-image annotations. In some cases, the same clinician 104 who provided the on-image annotations may also provide the questions and/or answers by operating one or more interfaces associated with training data generation module 110. In some embodiments, each annotated medical image (including questions, on-image labels and answers) may be reviewed by two other medical experts to achieve a high interrater reliability (e.g., 80% or higher). In some embodiments, the questions and/or answers may be linked to the on-image annotations. For example, in some embodiments, each on-image annotation may have a unique identifier that is connected to a question (e.g., 1A and 1E would be the first and fifth on-image annotations related to question #1).

In various embodiments, the training data 112 generated by training data generation module 110 may include (i) digital images, (ii) on-image annotations, and (iii) pairs of questions and answers (also referred to as “question-answer pairs”) in textual form. These training examples may be used, e.g., by training system 114, to train one or more machine learning models (which ultimately may be stored as trained models in database 116). Thereafter, Q&A system may use the trained models 116 to answer questions posed by patients about medical images (which may or may not include on-image annotations).

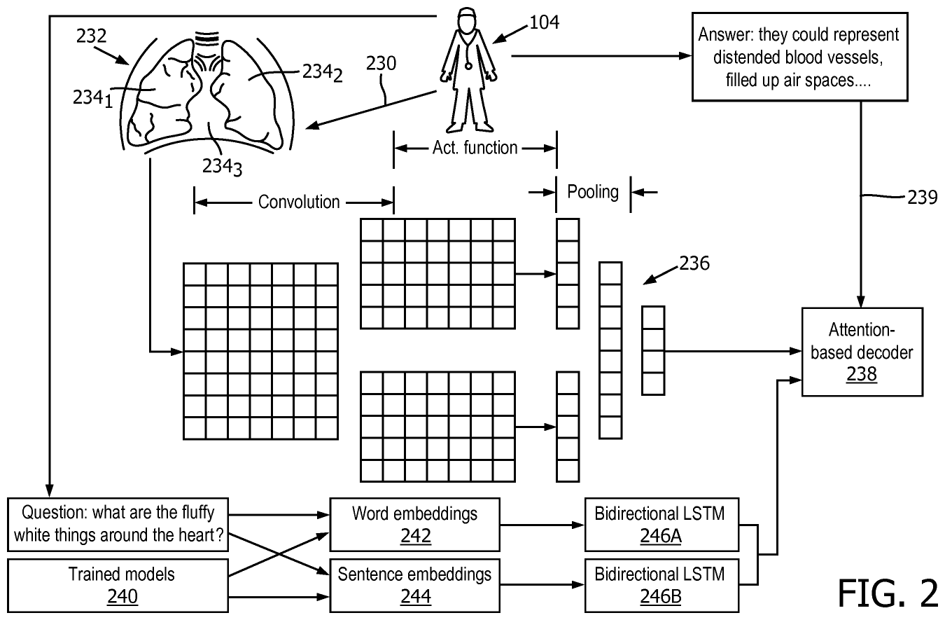

FIG. 2 schematically demonstrates one example of how machine learning models may be trained in order to facilitate VQA, in accordance with the present disclosure. At arrow 230, clinician 104 may formulate, for addition to a medical image 232, one or more on-image annotations 234₁₋₃. While represented in FIG. 2 simply as numbers, it should be understood that on-image annotations 234 may take various forms, such as textual labels, text accompanied by callout structures (e.g., arrows, brackets), dimensioning primitives (e.g., callouts designed to depict spatial dimensions), and so forth.

In FIG. 2, the annotated medical image 232 is provided as input to a machine learning model, along with a question (“What are the fluffy white things around the heart?”). In particular, medical image 232 itself is applied as input to an encoder portion 236 of an autoencoder, and the answer is applied s input to an attention-based decoder portion of 238 the autoencoder. As indicated in FIG. 2, in some embodiments, encoder portion 236 may include a convolutional portion (i.e. a CNN) with activation functions used for various layers (“ACT. FUNCTION” in FIG. 2), and a pooling portion. Encoder portion 236 may process the medical image 232 and its on-image annotations in order to learn and represent high-level features.

In some embodiments, the CNN portion of encoder portion 236 may include a text-attention layer that uses the on-image annotations for additional feature representation in a supervised manner. In some embodiments, on-image annotations may be analyzed to identify regions-of-interest in the medical image, e.g., regions that depict the medically-significant or interesting features that are called out by the on-image annotations. In some cases if an on-image annotation contains an arrow or another explicit call-out mechanism, the tip of the arrow may be used to identify one or more particular pixels and/or pixel coordinates. These pixel(s) and/or pixel coordinates may be expanded into a region of interest to ensure capture of the relevant medical feature. If no arrows or other obvious call out mechanisms are present (e.g., simply a textual label), then an area encompassing the textual label may be automatically or manually selected as a region of interest (e.g., expanding from one or more centrally-located pixel coordinates). In some embodiments, on-image annotations may be enhanced by contrast-based demarcation of regions to facilitate accurate representation and extraction of the pertinent features.

The regions of interest determined using the on-image annotations may be used as attention mechanisms to focus the CNN/encoder portion 236 on appropriate regions of medical image 232. In parallel, one or both of the question and answer posed by clinician 104 (or another expert using module 110) may be encoded using pre-trained models 240 (e.g., models developed using publicly available training data that includes, for instance, billions of words from online news articles) with word embeddings 242 and/or sentence embeddings 244 learned from (e.g., bidirectional) LSTMs 246A and 246, respectively. In some embodiments, these encodings, along with the encodings generated from the medical image 232, may be combined (e.g., concatenated) as input for attention-based decoder portion 238. Additionally or alternatively, in some embodiments, a single complex architecture having both a CNN layer and an LSTM/GRU layer may be employed.

In some embodiments, attention-based decoder portion 238 may be configured to recreate, or simply retrieve, the answer portion of the question-answer pair. Thus, once the whole neural network-based machine learning model is trained, decoder portion 238 may be configured to generate data indicative of an answer. In particular, and as described previously, decoder portion 238 may generate this data based on input in the form of the joint encoding that includes (i) encoded semantic features of a digital image about which a question is being posed, and (ii) encoded semantic concepts from the question posed about the digital image.

In some embodiments, attention-based decoder portion 238 may include a recurrent neural network (“RNN”). The RNN may learn and connect the output features from encoder portion 236 (e.g., the output features from the text-attentional CNN and question context embeddings) so that it can generate corresponding answers related to each digital image 232. In some embodiments, an attention component (e.g., one or more layers) of the RNN may be learned primarily from the feature representations of the digital image 232, each question-answer pair, and the on-image annotations of the training examples. Additionally or alternatively, in some embodiments (e.g., that are trained solely to handle “factoid” type questions), a similar neural network architecture may be employed, except that decoder portion 238 may instead be a relatively simpler feed-forward neural network that includes, for instance, a softmax layer to output the most probable answer to a question.

In some embodiments, the answer (“They could represent distended blood vessels, filled up air spaces, . . . ”) may be provided directly to decoder portion 238, as indicated by arrow 239 in FIG. 2 (which may be in addition to or instead of being provided to encoder portion 236). In some such embodiments, the answer may be persisted (e.g., stored in memory) verbatim. During training, the stored answer may be associated with the joint encoding of the image, on-image annotation(s), and question (and in some cases, the answer as well). Once trained, the answer may be retrievable based on output of decoder portion 238. For example, if a user later submits a semantically-similar image and question to Q&A system 118, the encoding generated from the user's question and image may be mapped, e.g., by the trained model, to the answer shown in FIG. 2.

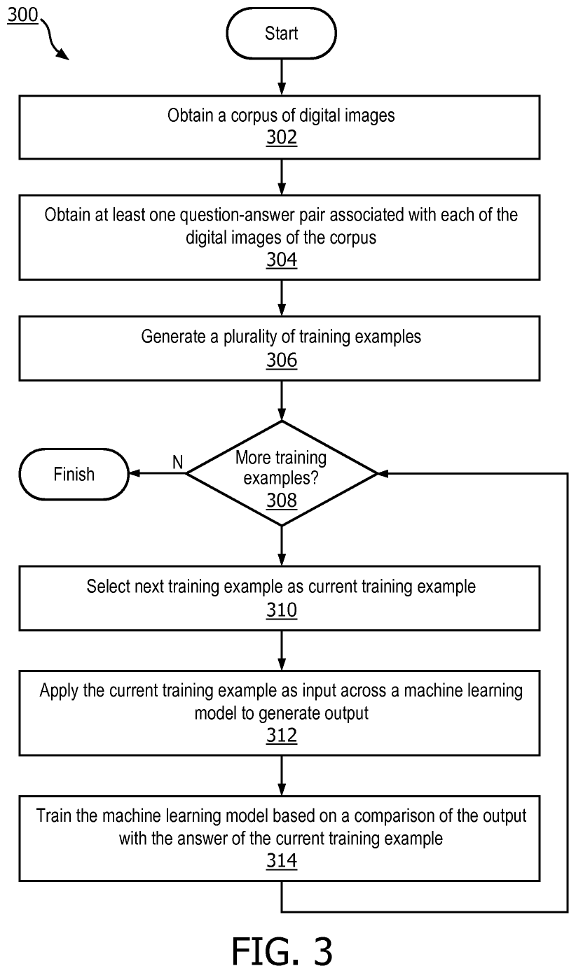

FIG. 3 depicts an example method 300 for training a machine learning model to perform VQA, in accordance with techniques described herein. For convenience, the operations of the flow chart are described with reference to a system that performs the operations. This system may include various components of various computer systems, including components depicted in FIG. 1. Moreover, while operations of method 300 are shown in a particular order, this is not meant to be limiting. One or more operations may be reordered, omitted or added.

At block 302, the system may obtain a corpus of digital images. In various embodiments, each respective digital image of the corpus may include one or more on-image annotations. And each on-image annotation may identify at least one pixel coordinate on the respective digital image. For example, if the annotation includes an arrow or other similar feature, the pixel(s) pointed to by the tip of the arrow may be the at least one pixel coordinate of the digital image.

At block 304, the system may obtain, e.g., from experts interacting with training data generation module 110, at least one question-answer pair associated with each of the digital images of the corpus. In some embodiments, as many as twenty or more questions and corresponding answers may be generated for a single digital image. At block 306, the system may generate a plurality of training examples. In some embodiments, each training example may include a respective digital image of the corpus, including the associated on-image annotations, and one or more associated question-answer pairs.

At block 308, a loop may begin by the system checking to see if there are any more training examples. If the answer is no, then method 300 may finish. However, if there are more training examples, then at block 310, the system may, at block 310, select a next training example as a “current” training example. Then, at block 312, the system may apply the current training example as input across a machine learning model to generate output. As noted above, in various embodiments, the machine learning model may include an encoder portion 236 and a decoder portion 238. The encoder portion 236 may include an attention layer that is configured to focus the encoder portion 236 on a region of the digital image of the current training example. In various embodiments, the region may be selected based on the at least one pixel coordinate identified by the on-image annotation of the digital image of the current training example.

At block 314, the system may train the machine learning model based on comparison of the output generated based on the machine learning model with the answer of the current training example. For example, in some embodiments, the difference, or error, may be used to perform operations such as stochastic gradient descent and/or back propagation to minimize a loss function and/or adjust weights and/or other parameters of encoder portion 236 and/or decoder portion 238.

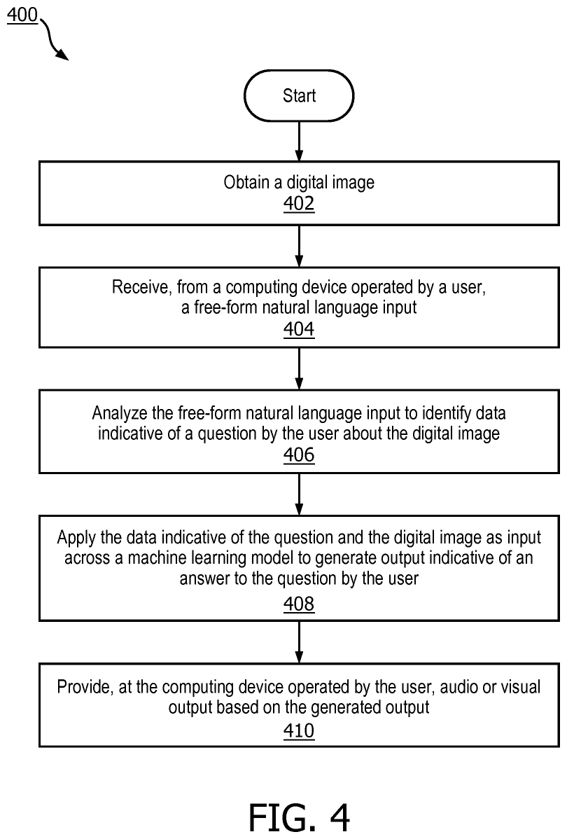

FIG. 4 depicts an example method 400 for practicing selected aspects of the present disclosure, particularly for applying a machine learning model trained using techniques such as method 300, in accordance with various embodiments. For convenience, the operations of the flow chart are described with reference to a system that performs the operations. This system may include various components of various computer systems, including various components of FIG. 1 such as Q&A system 118. Moreover, while operations of method 400 are shown in a particular order, this is not meant to be limiting. One or more operations may be reordered, omitted or added.

At block 402, the system may obtain a digital image, e.g., about which a user intends to ask a question. For example, a patient (e.g., 120) may operate a client device (e.g., 122) to navigate a web browser to a web portal associated with their healthcare provider, log in, and view one or more medical images of the patient's anatomy, e.g., CT scans, MRIs, x-rays, ultrasound images, etc. At block 404, the system, e.g., by way of Q&A system 118, may receive, from a computing device (e.g., 122) operated by the patient, a free-form natural language input. As a non-limiting example, the same web portal that provides the patient with access to health information may also include a chat bot interface that allows the patient to engage with a chat bot using natural language. In various embodiments, the patient may speak or type a question about an image the patient is current using. In some embodiments in which the patient speaks the natural language input question, speech-to-text (“STT”) processing may be performed, e.g., at Q&A system 118, to transform the patient's spoken utterance into textual content.

At block 406, the system may analyze the free-form natural language input to identify data indicative of a question by the user about the digital image. In the ongoing patient example, Q&A system 118 may include a natural language understanding engine (not depicted) that may annotate the question, identify salient semantic concepts and/or entities, and/or determine the patient's intent (i.e., identify the question).

At block 408, the system may apply the data indicative of the question (e.g., an intent and one or more slot values) and the digital image as input across a machine learning model trained using method 300 to generate output indicative of an answer to the question by the user. For example, in some embodiments, the output may be used to select an answer that was provided during training. For example, the output may include a plurality of candidate answers and corresponding probabilities that those candidate answers are correct.

At block 410, the system may provide, at the computing device operated by the user (e.g., 122 operated by patient 120), audio or visual output based on the generated output. For example, in the ongoing patient example, the chatbot may render, audibly and/or visually, natural language output that includes one or more semantic concepts/facts that are responsive to the patient's question.

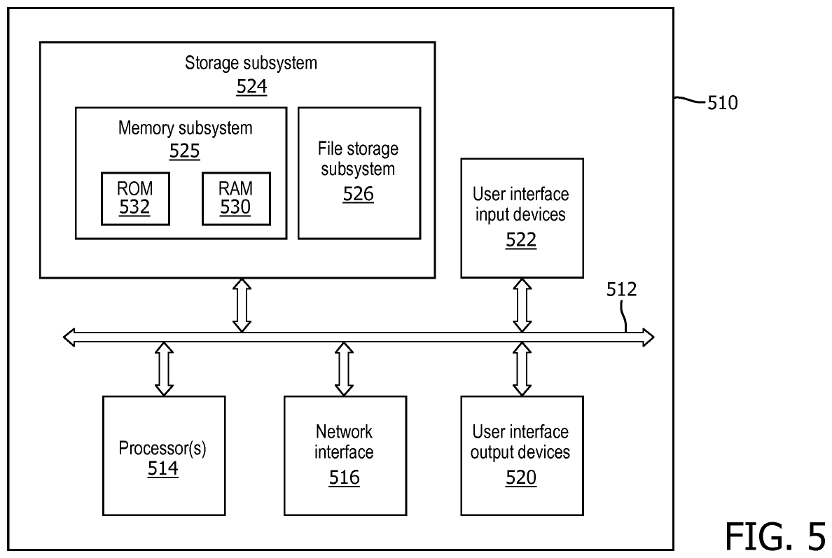

FIG. 5 is a block diagram of an example computing device 510 that may optionally be utilized to perform one or more aspects of techniques described herein. In some implementations, one or more of a client computing device, user-controlled resources engine 130, and/or other component(s) may comprise one or more components of the example computing device 510.

Computing device 510 typically includes at least one processor 514 which communicates with a number of peripheral devices via bus subsystem 512. These peripheral devices may include a storage subsystem 524, including, for example, a memory subsystem 525 and a file storage subsystem 526, user interface output devices 520, user interface input devices 522, and a network interface subsystem 516. The input and output devices allow user interaction with computing device 510. Network interface subsystem 516 provides an interface to outside networks and is coupled to corresponding interface devices in other computing devices.

User interface input devices 522 may include a keyboard, pointing devices such as a mouse, trackball, touchpad, or graphics tablet, a scanner, a touchscreen incorporated into the display, audio input devices such as voice recognition systems, microphones, and/or other types of input devices. In general, use of the term “input device” is intended to include all possible types of devices and ways to input information into computing device 510 or onto a communication network.

User interface output devices 520 may include a display subsystem, a printer, a fax machine, or non-visual displays such as audio output devices. The display subsystem may include a cathode ray tube (CRT), a flat-panel device such as a liquid crystal display (LCD), a projection device, or some other mechanism for creating a visible image. The display subsystem may also provide non-visual display such as via audio output devices. In general, use of the term “output device” is intended to include all possible types of devices and ways to output information from computing device 510 to the user or to another machine or computing device.

Storage subsystem 524 stores programming and data constructs that provide the functionality of some or all of the modules described herein. For example, the storage subsystem 524 may include the logic to perform selected aspects of the methods of FIGS. 3 and 4, as well as to implement various components depicted in FIGS. 1 and 2.

These software modules are generally executed by processor 514 alone or in combination with other processors. Memory 525 used in the storage subsystem 524 can include a number of memories including a main random access memory (RAM) 530 for storage of instructions and data during program execution and a read only memory (ROM) 532 in which fixed instructions are stored. A file storage subsystem 526 can provide persistent storage for program and data files, and may include a hard disk drive, a floppy disk drive along with associated removable media, a CD-ROM drive, an optical drive, or removable media cartridges. The modules implementing the functionality of certain implementations may be stored by file storage subsystem 526 in the storage subsystem 524, or in other machines accessible by the processor(s) 514.

Bus subsystem 512 provides a mechanism for letting the various components and subsystems of computing device 510 communicate with each other as intended. Although bus subsystem 512 is shown schematically as a single bus, alternative implementations of the bus subsystem may use multiple busses.

Computing device 510 can be of varying types including a workstation, server, computing cluster, blade server, server farm, or any other data processing system or computing device. Due to the ever-changing nature of computers and networks, the description of computing device 510 depicted in FIG. 5 is intended only as a specific example for purposes of illustrating some implementations. Many other configurations of computing device 510 are possible having more or fewer components than the computing device depicted in FIG. 5.

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03. It should be understood that certain expressions and reference signs used in the claims pursuant to Rule 6.2(b) of the Patent Cooperation Treaty (“PCT”) do not limit the scope.

1-20. (canceled)

21. A method implemented using one or more processors, comprising:
obtaining a digital image;
receiving, from a computing device operated by a user, a free-form natural language input;
analyzing the free-form natural language input to identify data indicative of a question by the user about the digital image;
applying the data indicative of the question and the digital image as input across a machine learning model to generate output indicative of a response to the question by the user; and
providing, at the computing device operated by the user, audio or visual output based on the generated output;
wherein the machine learning model includes an encoder portion and a decoder portion that are trained using a plurality of training examples, wherein each respective training example includes:
a digital image that includes one or more on-image annotations that are used to focus attention of the encoder portion on a region of the digital image; and
a question-answer pair associated with the digital image of the respective training example, wherein the question-answer pair includes a question and a corresponding answer;
wherein the decoder portion decodes the answer of the at least one question-answer pair of the respective training example based on an encoding generated using the digital image, the one or more on-image annotations, and at least the question of the question-answer pair of the respective training example as input.

22. The method of claim 21, wherein the encoder portion comprises a convolutional neural network.

23. The method of claim 21, wherein the decoder portion comprises a recurrent neural network.

24. The method of claim 21, wherein the encoder portion comprises one or more long short term memory networks.

25. The method of claim 21, wherein the encoder portion comprises one or more gated recurrent units.

26. The method of claim 21, wherein the corpus of digital images comprises medical images obtaining using one or more of magnetic resonance imaging, computed tomography scanning, and x-ray imaging, and the on-image annotations identify medically-significant features of the medical images.
